(19) United States
(12) Patent Application Publication
(10) Pub. No.: US 2006/0074352 A1
(43) Pub. Date: Apr. 6, 2006

(54) Wireguide with indicia

(76) Inventors: Brian C. Case, Bloomington, IN (US); Ram H. Paul, Bloomington, IN (US)

Correspondence Address: DUNLAP, CODDING & ROGERS, P.C., P.O. BOX 16370, OKLAHOMA CITY, OK, 73113, US

(21) Appl. No.: 11/244,846

(22) Filed: Oct. 6, 2005

Related U.S. Application Data
(60) Provisional application No. 60/616,234, filed on Oct. 6, 2004.

(51) Int. Cl. A61M 25/00

(52) U.S. Cl. 600/585

(57) ABSTRACT

Wireguides and methods of making wireguides for delivery systems are provided. A wireguide for a delivery system according to the invention facilitates placement of an intraluminal medical device at a desired point of treatment. The wireguides include a plurality of indicia, each of which has a configuration that differs from that of at least one other indicium of the plurality of indicia.

### CROSS-REFERENCE TO RELATED APPLICATIONS

This application claims priority to U.S. Provisional Application Ser. No. 60/616,234, filed on Oct. 6, 2004, which is hereby incorporated into this disclosure in its entirety.

### FIELD

Wireguides useful in the delivery and/or placement of intraluminal medical devices are disclosed.

### BACKGROUND

Minimally invasive techniques and instruments for placement of intraluminal medical devices have been developed over recent years and are frequently used to deliver and deploy an intraluminal medical device at a desired point of treatment. In these techniques, a delivery system is used to carry the intraluminal medical device through a body vessel along a wireguide to the point of treatment. Once the point of treatment is reached, the intraluminal medical device is deployed from the delivery system. The delivery system is subsequently withdrawn from the point of treatment and, ultimately, the body vessel. A wide variety of treatment devices that utilize minimally invasive technology has been developed and includes stents, stent grafts, occlusion devices, infusion catheters, prosthetic valves, and the like.

The wireguide is typically inserted in the body vessel and advanced through the body vessel to the vicinity of the point of treatment. The wireguide functions to guide the delivery system or other medical devices through the body vessel to the vicinity of the point of treatment. Typically, the delivery system or other medical device includes a lumen which is adapted to receive the wireguide therein.

It is desirable in many procedures to deploy an endoluminal medical device at a predetermined distance from a point of interest, such as an anatomical landmark. Accordingly, a need exists for medical devices that facilitate placement of an intraluminal medical device at a desired point of treatment.

### SUMMARY OF EXEMPLARY EMBODIMENTS

The invention provides wireguides that can be used during delivery of an intraluminal medical device to a point of treatment in a body vessel. Wireguides according to the invention facilitate placement of an intraluminal medical device at a desired point of treatment.

In one exemplary embodiment, a wireguide comprises an elongate member having proximal and distal ends. At least one indicium is formed adjacent the distal end of the wireguide. The at least one indicium is formed of a radiopaque material.

Delivery systems are also disclosed. In one exemplary embodiment, a delivery system according to the invention comprises an elongate tubular member having a distal end adapted for insertion into a body vessel. A dilator having a distal end adapted for insertion into the body vessel is disposed in the tubular member and includes a lumen formed therein. An intralumirial medical device is disposed radially between the tubular member and the dilator. An elongate wireguide having proximal and distal ends is insertable in the lumen of the dilator. The wireguide includes at least one indicium formed of a radiopaque material adjacent the distal end thereof.

The invention also provides methods of producing a wireguide. One method according to the invention comprises the steps of providing a wireguide and forming at least one indicium adjacent a distal end of the wireguide. The at least one indicium is formed of a radiopaque material.

### BRIEF DESCRIPTION OF THE DRAWINGS

FIG. 1 is a perspective view of a delivery system according to an embodiment of the invention.

FIG. 2 is a sectional view of the distal end of a delivery system illustrated in FIG. 1.

FIG. 3 is an enlarged elevational view of a wireguide of the delivery system illustrated in FIG. 1.

FIG. 4 is an enlarged elevational view of a wireguide according to another embodiment of the invention.

FIG. 5 is an enlarged elevational view of a wireguide according to another embodiment of the invention.

FIG. 6 is an enlarged elevational view of a wireguide according to another embodiment of the invention.

FIG. 7 is a sectional view of a body vessel within which the delivery system of FIG. 1 is inserted.

FIG. 8 is a flow diagram illustrating a method of producing a delivery system according to an embodiment of the invention.

### DETAILED DESCRIPTION OF EXEMPLARY EMBODIMENTS

The following detailed description and appended drawings describe and illustrate various exemplary embodiments. The description and drawings serve to enable one skilled in the art to make and use the invention, and are not intended to limit the scope of the invention, or its protection, in any manner.

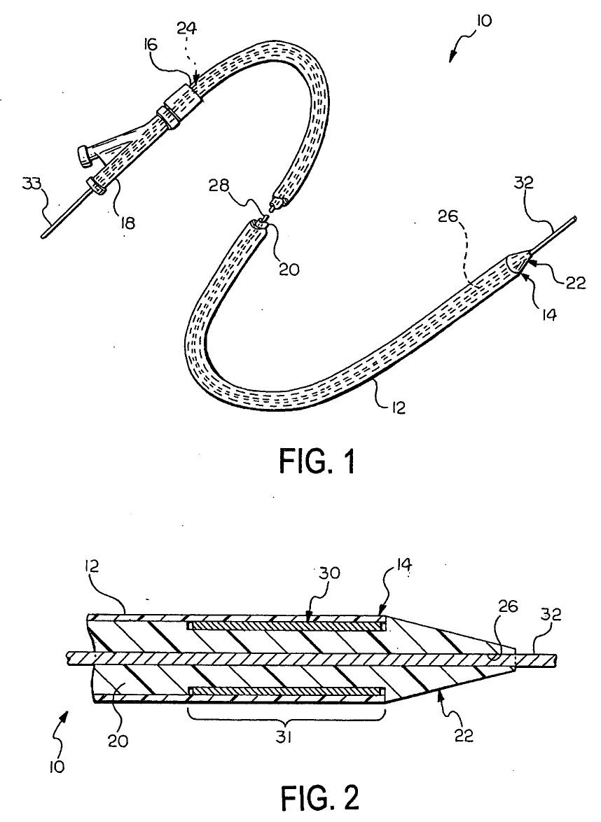

FIG. 1 illustrates a delivery system 10. The delivery system 10 includes an elongate sheath or tubular member 12 having a distal end 14, and a proximal end 16 that can be coupled to a connector 18 such as a Touhy Borst adapter, for example. The tubular member 12 is formed of any suitable flexible material, such as polyurethane or other polymeric material.

A dilator 20 is disposed within the tubular member 12. As used herein, the term “dilator” refers to an elongate member capable of being disposed within a lumen of a sheath, such as the tubular member 12. The dilator 20 has a tapered distal end 22 and a proximal end 24. A lumen 26 is formed by the dilator 20 and extends along the entire length of the dilator 20. The lumen 26 is adapted to receive a guiding member, such as a wireguide 28 or other suitable guiding member, therein. The lumen 26 may aid in guiding the delivery system 10 over the wireguide 28 to a desired point of treatment.

As used herein, the term “wireguide” refers to elongate members used in minimally invasive procedures to define a path along which other devices can be advanced. The term is considered equivalent in meaning to the term “guidewire” as also used in the art. The term does not require any particular material in the composition of the elongate member.

FIG. 2 illustrates the distal end of the delivery system 10 illustrated in FIG. 1. An intraluminal medical device 30 is shown disposed in a device chamber 31 formed in the dilator 20 adjacent the distal end 22 thereof. The intraluminal medical device 30 may be any suitable intraluminal medical device such as a stent, a prosthetic valve, a filter, an occlusion device, a distal protection device, a stent graft, and the like, for example. Wireguides and delivery systems according to the invention can be used with any type of endoluminal medical device, and the invention is not limited to any particular type of endoluminal medical device. Examples of suitable endoluminal medical devices for us in and with devices according to the invention include those described in U.S. Pat. No. 6,508,833 to Pavcnik et al. for MULITPLE-SIDED INTRALUMINA MEDICAL DEVICE; U.S. Pat. No. 6,464,720 to Boatman et al. for RADIALLY EXPANDABLE STENT; U.S. Pat. No. 6,231,598 to Berry et al. for a RADIALLY EXPANDABLE STENT; U.S. Pat. No. 6,299,635 to Frantzen for a RADIALLY EXPANDABLE NON-AXIALLY CONTRACTING SURGICAL STENT; U.S. Pat. No. 5,580,568 to Gianturco for a PERCUTANEOUS ENDOVASCULAR STENT AND METHOD FOR INSERTION THEREOF; and published application for United States patent 20010039450 to Pavcnik et al. for an IMPLANTABLE MEDICAL DEVICE, all of which are hereby incorporated by reference in their entirety for the purpose of describing suitable endoluminal medical devices for use in and with devices according to the invention.

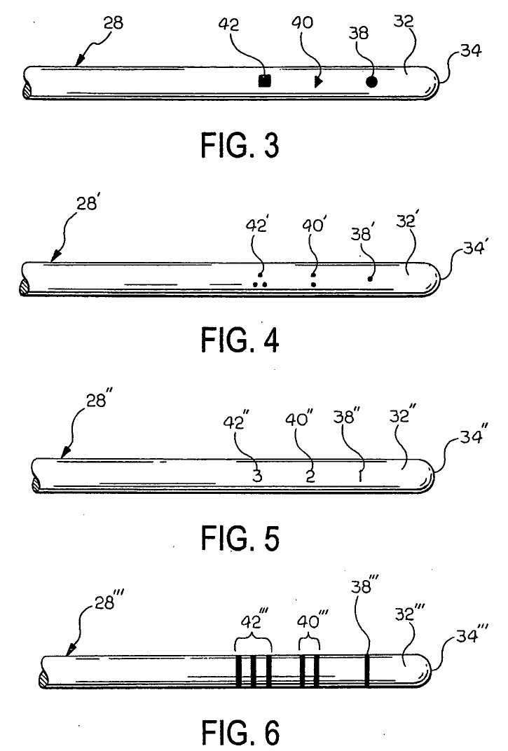

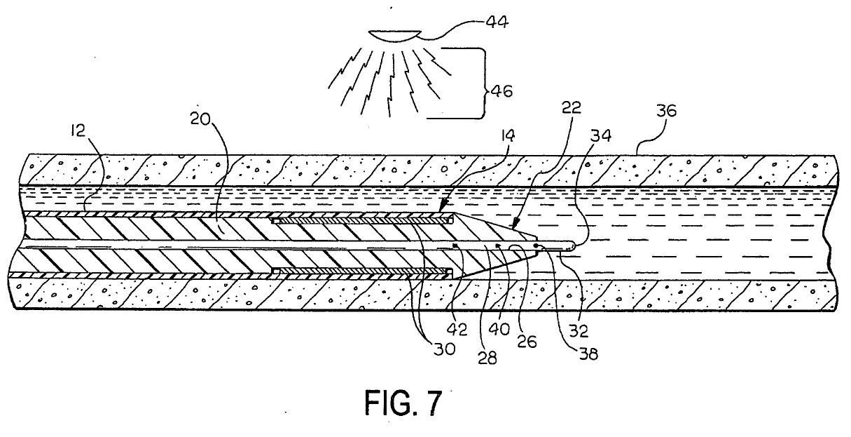

FIG. 3 shows the distal end 32 of the wireguide 28 illustrated in FIGS. 1 and 2. The proximal end 33 of the wireguide 28 is shown in FIG. 1. A tip 34 of the distal end 32 of the wireguide 28 has a partial spherical shape to facilitate insertion of the wireguide 28 in a body vessel 36, as illustrated in FIG. 7. It is understood that other shapes can be used as desired, including a substantially conical shape, for example.

A plurality of indicia are formed on the distal end 32 of the wireguide 28. As described below, each indicium of the plurality of indicia is positioned at a particular point on the wireguide 28 and provides an indication of that particular point. Each of the indicia can have any suitable configuration. Also, each indicium has a configuration that is different that the configuration of at least one other indicium of the plurality of indicia. Optionally, each indicium of the plurality of indicia has a configuration that is different than the configuration of all other indicia in the plurality of indicia. This arrangement allows a user to distinguish one indicium from the others, which aids in use of devices according to the invention. Prior art devices, in which each indicium has the same configuration, make it difficult, if not impossible, to distinguish one indicium from another limiting the effectiveness of these devices. As used in this context, the term “configuration” refers to the arrangement of the element(s) of the indicium that are viewable using appropriate imaging techniques and apparatuses.

In the embodiment illustrated in FIG. 3, the wireguide 28 includes first 38, second 40, and third indicia 42. More or fewer indicia can be used as desired. The first indicium 38 is spaced a predetermined distance from the tip 34 of the distal end 32 of the wireguide 28. It is understood that an indicium can be positioned at the tip 34 of the distal end 32. The second indicium 40 is spaced a predetermined distance from the first indicium 38, and the third indicium 42 is spaced a predetermined distance from the second indicium 40. Additional indicia can be spaced from the third indicium 42 a predetermined distance as desired, and continuing with additional indicia. In the embodiment shown, the predetermined distance from the tip 34 to the first indicium 38, the predetermined distance from the first indicium 38 to the second indicium 40, and the predetermined distance from the second indicium 40 to the third indicium 42 are equivalent. It is understood, though, that some or all of the predetermined distances can be varied as desired. The specific predetermined distances chosen for any particular device according to the invention will depend upon several considerations, including the intended use of the device.

Each of the indicia 38, 40, 42 on the wireguide 28 according to the embodiment shown in FIG. 3 has a configuration that differs from the configuration of each of the other indicia. In this example, each indicium 38, 40, 42 has a unique geometric shape. As illustrated in FIG. 3, the shapes used for the indicia 38, 40, 42 are a circle, a triangle, and a square, respectively. It is understood, though, that other shapes can be used for the indicia 38, 40, 42 as desired. The use of different geometrical shapes is considered advantageous at least because the shapes are dramatically different from one another decreasing the likelihood that one indicium would be confused for another under visualization.

Any conventional material can be used to form the indicia 38, 40, 42. A radiopaque material is advantageously used. These materials are known in the art and facilitate use of devices according to the invention with visualization techniques, such as fluoroscopy and x-ray techniques. The indicia 38, 40, 42 can be formed of any conventional radiopaque material such as barium sulfate, bismuth salts, bismuth subcarbonate, tungsten, or tungsten powder, for example. Also, the indicia 38, 40, 42 can be formed in, with, or on the wireguide 28. The different configurations can be formed using various suitable techniques, such as masking.

FIG. 4 shows the distal end 32′ of the wireguide 28′ according to another exemplary embodiment. Similar structure from FIGS. 1 through 3 is represented by the same reference numeral and a prime (′) symbol. The first 38′, second 40′, and third indicia 42′ are formed on the wireguide 28′. More or fewer indicia 38′, 40′, 42′ can be used as desired. The first indicium 38′ is spaced a predetermined distance from the tip 34′ of the distal end 32′ of the wireguide 28′. It is understood that an indicium can be positioned at the tip 34′ of the distal end 32′. The second indicium 40′ is spaced a predetermined distance from the first indicium 38′ and the third indicium 42′ is spaced a predetermined distance from the second marking 40′. Additional indicia can be spaced from the third indicium 42′ by a predetermined distance as desired, and continuing with additional indicia. In the embodiment shown, the predetermined distance from the tip 34′ to the first indicia 38′, the predetermined distance from the first indicia 38′ to the second indicia 40′, and the predetermined distance from the second indicia 40′ to the third indicia 42′ are equivalent. It is understood, though, that some or all of the predetermined distances can be varied as desired. Similar to the different shapes illustrated in FIG. 3, the different grouping of dots in this example are considered advantageous at least because each grouping is dramatically different from each of the other groupings, decreasing the likelihood that one indicia would be confused for another under visualization. If the groupings are placed in a numerical order, such as that illustrated in FIG. 4, an additional advantage is provided in that a logical order is provided without requiring additional training to a user.

Each indicium 38′, 40′, 42′ in the example illustrated in FIG. 4 comprises a grouping of dots. The first indicia 38′ comprises a single dot, second indicia 40′ comprises two dots, the third indicia 42′ comprises three dots, and so on. Thus, each indicium 38′, 40′, 42′ has a configuration that differs from that of each of the other indicia. The dots are arranged to represent numerical positions increasing sequentially beginning at the distal end 32′ and moving towards the proximal end 33′. Other arrangements or quantities of dots can be used as desired. Similar to the different shapes illustrated in FIG. 3, the different grouping of dots in this example is considered advantageous at least because each grouping is dramatically different from each of the other groupings, decreasing the likelihood that one indicia would be confused for another under visualization. If the groupings are placed in a numerical order, such as that illustrated in FIG. 4, an additional advantage is provided in that a logical order is provided without requiring additional training to a user.

FIG. 5 shows the distal end 32″ of the wireguide 28″ according to another embodiment of the invention. Similar structure from FIGS. 1-4 is represented by the same reference numeral and a double prime (″) symbol. The first 38″, second 40″, and third indicia 42″ are formed on the wireguide 28″. More or fewer indicia 38″, 40″, 42″ can be used as desired. The first indicia 38″ is spaced a predetermined distance from the tip 34″ of the distal end 32″ of the wireguide 28″. It is understood that an indicium can be positioned at the tip 34″ of the distal end 32″. The second indicium 40″ is spaced a predetermined distance from the first indicium 38″ and the third indicium 42″ is spaced a predetermined distance from the second indicium 40″. Additional indicia can be spaced from the third indicium 42″ a predetermined distance as desired, and continuing with additional indicia. In the embodiment shown, the predetermined distance from the tip 34″ to the first indicium 38″, the predetermined distance from the first indicium 38″ to the second indicium 40″, and the predetermined distance from the second indicium 40″ to the third indicium 42″ are equivalent. It is understood that some or all of the predetermined distances can be varied as desired.

In this exemplary embodiment, each indicium 38″, 40″, 42″ comprises an Arabic numeral. The first indicium 38″ is the numeral 1, the second indicium 40″ is the numeral 2, the third indicium 42″ is the numeral 3, and so on. Thus, each indicium 38″, 40″, 42″ has a configuration that differs from that of each of the other indicia. The numerals are arranged to increase sequentially beginning at the distal end 32″ and moving towards the proximal end 33″. Other types of numerals, including Roman numerals, can also be used. Similar to the previous examples, the different Arabic numerals are considered advantageous at least because each numeral is dramatically different than each other numeral, decreasing the likelihood that one indicium would be confused for another under visualization. Also, if the numerals are placed in a numerical order, such as that illustrated in FIG. 5, an additional advantage is provided in that a logical order is provided without requiring additional training to a user. Letters can also be employed, and any suitable logical orders, such as alphabetical recitations and spellings of names, places, words, letters, model numbers, trade names, or other suitable representations, can be used.

FIG. 6 shows the distal end 32′″ of the wireguide 28′″ according to another embodiment of the invention. Similar structure from FIGS. 1 through 5 is represented by the same reference numeral and a triple prime (′″) symbol. First 38′″, second 40′″, and third indicia 42′″ are formed on the wireguide 28′″. More or fewer indicia 38′″, 40′″, 42′″ can be used as desired. The first indicium 38′″ is spaced a predetermined distance from the tip 34′″ of the distal end 32′″ of the wireguide 28′″. It is understood that an indicium can be positioned at the tip 34′″ of the distal end 32′″. The second indicium 40′″ is spaced a predetermined distance from the first indicium 38′″ and the third indicium 42′″ is spaced a predetermined distance from the second indicium 40′″. Additional indicia can be spaced from the third indicium 42′″ a predetermined distance as desired, and continuing with additional indicia. In the embodiment shown, the predetermined distance from the tip 34′″ to the first indicium 38′″, the predetermined distance from the first indicium 38′″ to the second indicium 40′″, and the predetermined distance from the second indicium 40′″ to the third indicium 42′″ are equivalent It is understood that some or all of the predetermined distances can be varied as desired.

Each indicium 38′″, 40′″, 42′″ in the example illustrated in FIG. 6 comprises a set of spaced apart annular rings. The first indicium 38′″ comprises a single ring, the second indicium 40′″ comprises two rings, the third indicium 42′″ comprises three rings, and so on. Thus, each indicium 38′″, 40′″, 42′″ has a configuration that differs from that of each of the other indicia. The rings are arranged to represent numerical positions increasing sequentially beginning at the distal end 32′″ and moving towards the proximal end 33′″. Other arrangements or quantities of rings can be used as desired. Additionally, the rings shown are continuous around the outer surface of the wireguide 28′″. It is understood that an annular array of indicia forming a ring can be used. This embodiment is advantageous since the annular position of the indicia need not be taken into account during use of the wireguide 28′″.

Similar to the previous examples, the different sets of spaced apart annular rings are considered advantageous at least because each set is dramatically different than each other set, decreasing the likelihood that one indicium would be confused for another under visualization. Also, if the sets are placed in a logical order, such as the increasing numerical order illustrated in FIG. 6, an additional advantage is provided in that a logical order is provided without requiring additional training to a user.

FIG. 7 shows the delivery system 10 of FIGS. 1 through 3 inserted in a body vessel 36. The delivery device 10 illustrated in FIG. 7 is illustrated with the wireguide 28 shown in FIG. 3 for exemplary purposes. It is understood that a delivery system can include any wireguide according to the invention. A visualization source 44 such as a fluoroscopy or X-ray source, for example, is directing energy 46 at the portion of the body vessel 36 where the distal end of the delivery system 10 is located. Since the indicia 38, 40, 42 are detectable by the visualization source 44, a user can determine a position of the wireguide 28 within the body vessel 36 of a patient. Additionally, progress through the body vessel 36 of the wireguide 28 can be tracked using the visualization source 44. Thus, a desired point of treatment can be reached using the indicia 38, 40, 42 in conjunction with the fluoroscopy source 44. For example, if the intraluminal medical device 30 is a prosthetic valve and it is desired to place the prosthetic valve a predetermined distance from a particular anatomical marker in the body vessel 36, such as a natural valve, the distal end 34 of the wireguide 28 or one of the indicia 38,40,42 can be positioned at the anatomical marker. A dilator 20 carrying an endoluminal medical device 30 can be advanced over the wireguide 28 until a portion, such as an end, of the device 30 reaches an indicium 38, 40, 42 that corresponds to the desired distance. Once this point is reached, the endoluminal device 30 can be deployed, such as by retraction of the tubular member 12, at the desired distance from the anatomical marker.

Additionally, the indicia 38, 40, 42 can be used to measure or calculate a distance within the body vessel 36 using the visualization source 44. An axial position of the intraluminal medical device 30 can also be determined by observing and recording the position of the intraluminal medical device 30 with respect to the indicia 38, 40, 42 outside of the patient's body, then using that known position to deploy the intraluminal medical device 30 at a desired position in the body vessel 36 using the visualization source 44.

It should be noted that a distance between the indicia 38, 40, 42 can be taken at any point, as desired, on the indicia 38, 40, 42. For example, the distance can be measured from a midpoint of the indicia 38, 40, 42, or any other point, as desired. Additionally, narrow indicia (not shown) can be placed above, below, or above and below the indicia 38, 40, 42 to assist a user in determining the measuring point. Such narrow indicia can be similar to that used on a tape measure, for example. Additional intermediate indicia (not shown) can also be positioned between the indicia 38, 40, 42 to assist a user in determining incremental measurements between the indicia 38, 40, 42. It is understood that similar methods and additional indicia can be used with the other embodiments of the invention such as the arrangement of dots indicia 38′, 40′, 42′; the Arabic numeral indicia 38″, 40″, 42″; and the annular ring indicia 38′″, 40′″, 42′″. For example, with the annular ring indicia 38′″, 40′″, 42′″, the point of measurement can be to the first annular ring in the group of rings, or to the center of the group of rings, as desired.

The indicia 38, 40, 42 can also be used in conjunction with indicia (not shown) formed on the tubular member 12, the dilator 12, or the medical device 30. This indicia and the indicia 38, 40, 42 formed adjacent the distal end 32 of the wireguide 28 cooperate to assist the user in positioning of the medical device 30 based on positioning of the wireguide 28 relative to the tubular member 12, the dilator 20, or the medical device 30, for example.

Assembly of the delivery system 10 as herein described applies to each of the embodiments shown. The order of the steps is exemplary in nature and is not necessary or critical, and the description of the steps in an order is not intended to limit the invention in any manner. The intraluminal medical device 30 is disposed about the dilator 20 adjacent the distal end 22 thereof. The dilator 20 is inserted into the tubular member 12 to be substantially concentric therewith. The dilator 20 is inserted until the intraluminal medical device 30, the dilator 20, and the tubular member 12 are in the configuration shown in FIGS. 1 and 2. The tubular member 12 and the dilator 20 cooperate to maintain proper positioning of the intraluminal medical device 30 in the delivery system 10. Typically, the wireguide 28 is inserted into the lumen 26 formed in the dilator 20 during insertion of the distal end 22 of the dilator 20 into the body vessel 36.

Use of the delivery system 10 as herein described applies to each of the embodiments shown. The order of the steps is exemplary in nature and is not necessary or critical, and the description of the steps in an order is not or intended to limit the invention in any manner. In use, the delivery system 10 delivers the intraluminal medical device 30 to a desired location within the body vessel 36. To deliver the intraluminal medical device 30, the wireguide 28 is placed in the body vessel 36 of the patient by navigating the distal end 32 of the wireguide 28 to the desired area of treatment. The proximal end 33 of the wireguide 28 is left outside the body of the patient.

When it is desired to insert the delivery system 10 in the body vessel 36, the proximal end 33 of the wireguide 28 is inserted into the lumen 26 of the dilator 20 at the distal end 22. The distal end 22 of the dilator 20 is caused to enter the body vessel 36 along the wireguide 28 and is moved to the desired area of treatment. Typically, deployment of the intraluminal medical device 30 at the desired area of treatment can be accomplished by causing the intraluminal medical device 30 and the distal end 22 of the dilator 20 to be slidingly moved out of the tubular member 12, such as by retraction of the tubular member 12.

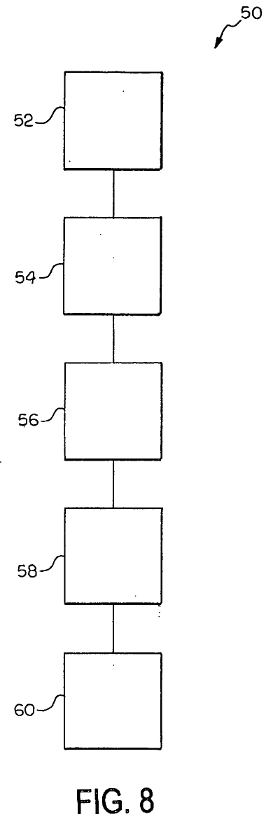

FIG. 8 provides a flow diagram illustrating a method 50 of producing the delivery system 10 according to the invention. The method 50 as herein described applies to each of the embodiments shown. The steps presented are exemplary in nature, and thus, the order is not necessary or critical. In one step 52, an elongate member having proximal and distal ends are provided. The elongate member may be the wireguide 28, for example. At least one indicium 38, 40, 42 is formed in, with, or on the wireguide 28, represented by 54. In the embodiment shown, the indicia 38, 40, 42 are formed in, with, or on the wireguide 28 a predetermined distance apart to facilitate observation thereof in the body of the patient and positioning of the wireguide 28. In another step 56, the dilator 20 is inserted in the tubular member 12 to be substantially coaxial therewith. The intraluminal medical device 30 is positioned adjacent the distal end 14 of the tubular member 12 radially between the tubular member 12 and the dilator 20, represented by 58. In another step 60, the assembly including the tubular member 12, the dilator 20, and the intraluminal medical device 30, is packaged with the wireguide 28 including the indicia 38, 40, 42. The assembly and the wireguide 28 can be packaged separately or together, as desired. It is understood that all steps can be performed in any order. The method can include any further steps necessary to produce a desired finished delivery system 10, such as attaching any suitable connectors, adapters, and the like on various components of the delivery system 10.

The foregoing detailed description provides exemplary embodiments of the invention and includes the best mode for practicing the invention. These embodiments are intended only to serve as examples of the invention, and not to limit the scope of the invention, or its protection, in any manner.

What is claimed is:

1. A wireguide, comprising:
an elongate member having a proximal end and a distal end; and
a plurality of indicia disposed on the distal end of the elongate member, each indicium of the plurality of indicia formed of a radiopaque material and having a configuration that is different than a configuration of at least one other indicium of the plurality of indicia.

2. The wireguide according to claim 1, wherein the configuration of each indicium of the plurality of indica is a geometric shape.

3. The wireguide according to claim 2, wherein the configuration of each indicium of the plurality of indicia is a unique geometric shape.

4. The wireguide according to claim 1, wherein the configuration of each indicium of the plurality of indicia comprises an arrangement of dots.

5. The wireguide according to claim 4, wherein the configuration of each indicium of the plurality of indicia is a unique arrangement of dots.

6. The wireguide according to claim 5, wherein the plurality of indicia are axially positioned on the elongate member in an order based on the number of dots present in each indicium.

7. The wireguide according to claim 6, wherein the indicium comprising either the least or most dots is positioned distal to all other indicia in the plurality of indicia.

8. The wireguide according to claim 6, wherein the indicia are positioned on the elongate member in an order according to the number of dots in a configuration beginning at the distal end and moving towards the proximal end of the elongate member.

9. The wireguide according to claim 8, wherein the order comprises a sequence in which the number of dots in a particular configuration increases as the indicia move progressively farther from the distal and of the elongate member.

10. The wireguide according to claim 1, wherein the configuration of each indicium of said plurality of indicia comprises a numeral.

11. The wireguide according to claim 10, wherein the indicia are axially positioned on the elongate member in an order according to the numerals represented by the configurations of the indicia.

12. The wireguide according to claim 11, wherein the order comprises a sequence in which the numerals increase as the indicia move progressively farther from the distal end of the elongate member.

13. The wireguide according to claim 1, wherein the configuration of each indicium of said plurality of indicia comprises a set of spaced apart annular rings.

14. The wireguide according to claim 13, wherein the indicia are axially positioned on the elongate member to represent numerical positions increasing sequentially beginning at the distal end and moving toward the proximal end of the elongate member.

15. A wireguide comprising:
an elongate member having a proximal end and a distal end; and
a plurality of radiopaque indicia formed on the distal end of the elongate member, each indicium of the plurality of indicia having a unique configuration among all other indicia of the plurality of indicia.

16. The wireguide according to claim 15, wherein the configuration of each indicium of the plurality of indica is a geometric shape.

17. The wireguide according to claim 15, wherein the configuration of each indicium of the plurality of indicia comprises an arrangement of dots.

18. The wireguide according to claim 15, wherein the configuration of each indicium of said plurality of indicia comprises a numeral.

19. The wireguide according to claim 15, wherein the configuration of each indicium of said plurality of indicia comprises a set of spaced apart annular rings.

20. A wireguide comprising:
an elongate member having a proximal end and a distal end and a plurality of indica formed on the distal end;
wherein each indicium of the plurality of indicia has a unique configuration that comprises a geometric shape.
